(19) United States
(12) Patent Application Publication
(10) Pub. No.: US 2011/0118786 A1
(43) Pub. Date: May 19, 2011

(54) SPINAL ROD SYSTEM

(76) Inventor: Jee-Soo Jang, Seoul (KR)

(21) Appl. No.: 13/000,334

(22) PCT Filed: Nov. 4, 2009
(86) PCT No.: PCT/KR09/06456

§ 371 Date: Jan. 24, 2011

(30) Foreign Application Priority Data
Nov. 14, 2008 (KR) 10-2008-0113630
Aug. 18, 2009 (KR) 10-2009-0076325

(51) Int. Cl. A61B 17/70

(52) U.S. Cl. 606/264

(57) ABSTRACT

Provided is a spinal rod system for simply and safely fixing a damaged vertebra by connecting the spinal rod system to a vertebra body fixing rod fixed to a vertebra through the prior procedure. The spinal rod system includes a connecting rod including a first curved part and a second curved part so that one end part and another end part are spaced apart from each other and parallel to each other, and a connecting unit for connecting the one end part of the connecting rod to the vertebra fixing rod fixed to the vertebra through the prior procedure.

### TECHNICAL FIELD

The present invention relates to a spinal rod system (i.e., connecting rod for spinal fixation), and more particularly, to a spinal rod system for simply and safely fixing a vertebra when the vertebra is additionally damaged after a prior procedure, by connecting the spinal rod system to a vertebra fixing rod fixed to the vertebra through the prior procedure.

### BACKGROUND ART

A spine may be damaged due to various reasons, such as a fracture of a vertebra or damage of an intervertebral disc due to an accident, or thickened facet joints due to aging. A patient with a damaged spine is initially treated with medication, an exercise cure, injections, a physical therapy, or the like. However, if symptoms are severe, a spine insert for maintaining an interval between vertebrae may be inserted between the vertebrae or a surgery of fixing a location of a damaged vertebra may be performed.

Here, a vertebra fixing surgery uses a pedicle screw. The vertebra fixing surgery is performed when a spine is unstably fractured (i.e., when all three columns of a spine are damaged), when a spine is deformed by 30° or above kaphotic angle or 40% or above compression rate or a spinal canal invasion is equal to or above 50% due to burst fracture, when damage to an entire structure of a posterior ligament complex is determined via magnetic resonance imaging (MRI), when evident neurological damage including muscle weakness is accompanied, or when a kaphotic angle accompanying acute pain or a neurological symptom is progressed despite of a suitable symptomatic treatment. The vertebra fixing surgery is also performed when there is evident neurological damage due to osteoporotic fracture, or when realignment is required since severe pain is continued and a spine is deformed despite of another suitable treatment. The vertebra fixing surgery may also be performed when curvature of 10° or above is determined via an inclinometer or a rib hump of 3 cm or above is determined via a rib hump measurer due to idiopathic scoliosis, or when scoliosis of 25° or above, lordosis of 20° or below, or evident subluxation is determined on a radioautograph due to degenerative scoliosis.

FIG. 1 is a diagram of a spine on which such a conventional vertebra fixing method is performed. Referring to FIG. 1, a vertebra fixing apparatus 110 for fixing a location of a vertebra includes a pedicle screw 112 that is implanted into a transverse process 100, a rod 114 for longitudinally connecting the pedicle screws 112, and a transverse bar 116 for transversely connecting the rods 114.

A method of fixing a vertebra by using the vertebra fixing apparatus 110 is as follows.

A part of patient's back to undergo a surgery is first incised, a damaged disc that is pressing a nerve is removed according to a state of the patient, and a spine is restored by putting an artificial spacer (not shown) instead of the damaged disc. Then, the pedicle screws 112 are implanted into the transverse processes 100 of vertebrae to be fixed. The implanted pedicle screws 112 are longitudinally disposed, and are connected to each other by the rods 114 inserted into each head unit (not shown) of the pedicle screws 112. The rods 114 are fixed by nuts combined to the head units of the pedicle screws 112. Here, a transverse connector 120 is built in the rod 114, and the transverse bar 116 is fixed at each transverse connector 120, thereby fixing the vertebrae.

As such, the vertebrae are fixed as the head units of the pedicle screws 112 fixed along the segmented vertebrae at each side of the traverse process 100 of the vertebrae are connected to each other by the rod 114.

Meanwhile, referring to FIG. 2, after a vertebra is fixed, when another vertebra that is on an extension of the vertebra is newly damaged, new pedicle screws 154 are driven into transverse processes 150 of the vertebra into which pedicle screws 152 are pre-driven via a prior procedure, and pedicle screws 156 are driven into the other newly damaged vertebra that is on the extension, thereby fixing the other newly damaged vertebra by connecting the pedicle screws 154 and 156 by using a rod 160.

However, according to such a conventional method, an area of the transverse process 150 of the vertebra, into which the new pedicle screws 154 may be driven, is small and thus may be too much. Also, a surgery time may lengthen due to an operation of newly driving the pedicle screws 154, and stability of the fixation of the other newly damaged vertebra may deteriorate.

### DISCLOSURE OF THE INVENTION

The present invention also provides a spinal rod system for simply and safely fixing an additionally damaged vertebra by connecting the spinal rod system to a vertebra fixing rod fixing a vertebra through a prior procedure.

According to an aspect of the present invention, there is provided a spinal rod system including: a connecting rod including a first curved part and a second curved part so that one end part and another end part are spaced apart from each other and parallel to each other; and a connecting unit for connecting the one end part of the connecting rod to a vertebra fixing rod fixed to a vertebra through a prior procedure.

The connecting unit may include: a pair of coupling parts formed in a single body to accommodate the connecting rod and the vertebra fixing rod; and a pair of nuts coupled to the coupling part to fix the connecting rod and the vertebra fixing rod.

### BRIEF DESCRIPTION OF THE DRAWINGS

FIG. 1 is a diagram of a spine on which a vertebra fixing method is performed;

FIG. 2 is a diagram of a spine on which a conventional vertebra fixing method due to additional damage;

FIG. 3 is a perspective view of a spinal rod system according to an embodiment of the present invention;

FIG. 4 is an exploded perspective view of a spinal rod system according to an embodiment of the present invention;

FIG. 5 is a plan view of a spinal rod system according to an embodiment of the present invention;

FIGS. 6A through 6C are diagrams sequentially illustrating connecting processes of a spinal rod system, according to an embodiment of the present invention; and

FIG. 7 is a diagram of a spinal rod system fixing a vertebra, according to an embodiment of the present invention.

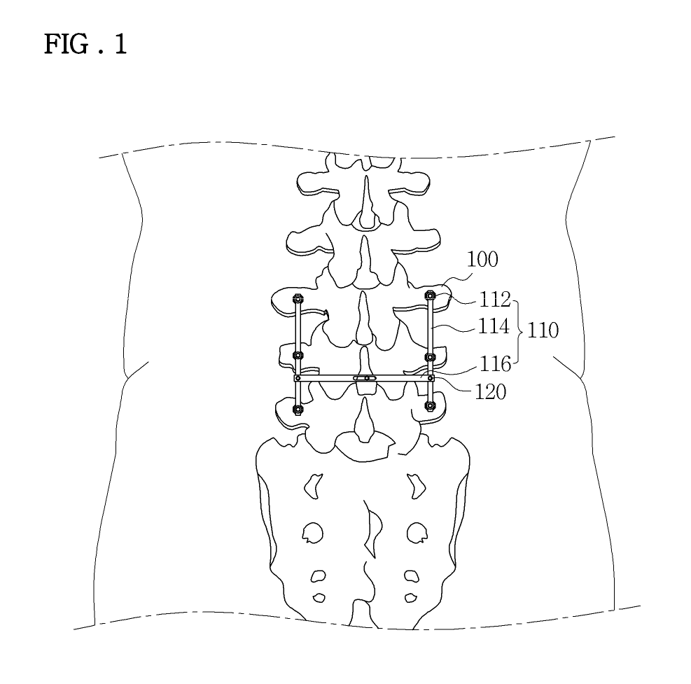

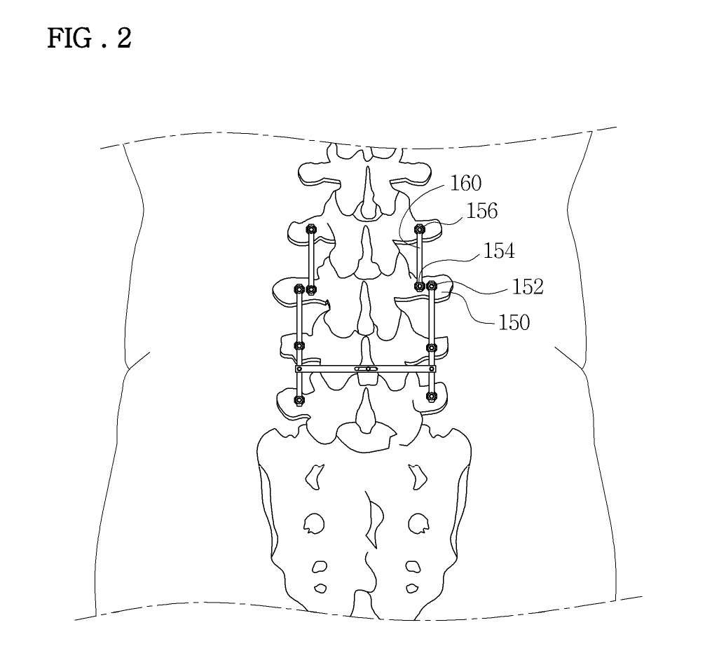

### BEST MODE FOR CARRYING OUT THE INVENTION

Hereinafter, a spinal rod system according to exemplary embodiments of the present invention will now be described with reference to attached drawings.

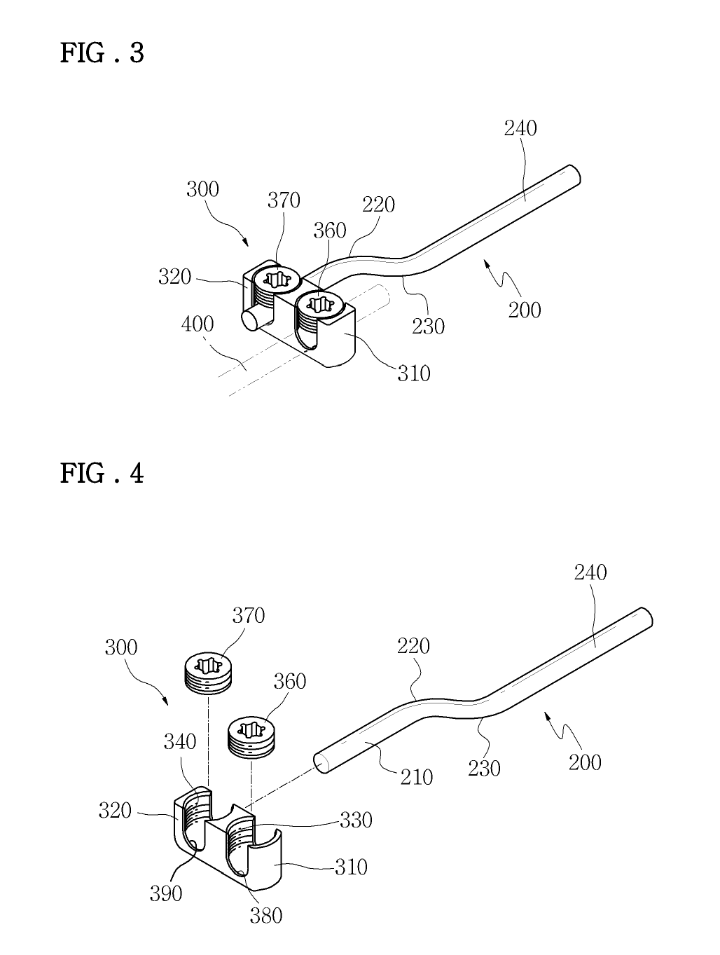

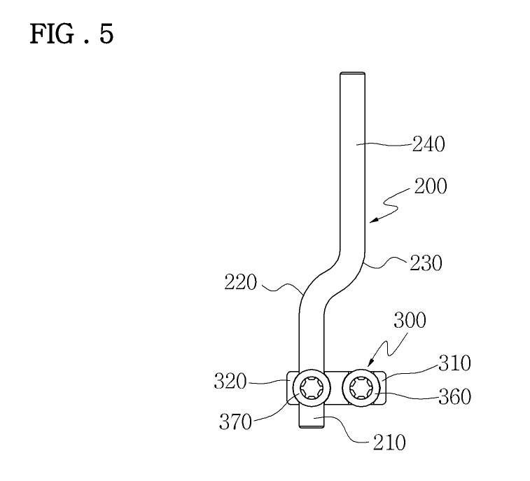

FIG. 3 is a perspective view of a spinal rod system according to an embodiment of the present invention, FIG. 4 is an exploded perspective view of the spinal rod system according to an embodiment of the present invention, and FIG. 5 is a plan view of the spinal rod system according to an embodiment of the present invention.

Referring to FIGS. 3 through 5, the spinal rod system includes a connecting rod 200 and a connecting unit 300.

In the connecting rod 200, one end part 210 and another end part 240 are spaced apart from and parallel to each other as a straight rod, i.e., the connecting rod 200, is bent at a predetermined location (a first curved part 220), and again bent in a direction opposite to a bent direction of the first curved part 220 at a location away from the first curved part 220 by a predetermined distance (a second curved part 230). Here, the connecting rod 200 is bent at a location so that the connecting rod 200 does not contact an end of a vertebra fixing rod 400 fixing a vertebra via a prior procedure.

The connecting unit 300 includes a pair of coupling parts (a first coupling part 310 and a second coupling part 320) that are formed in a single body, and a pair of nuts (a first nut 360 and a second nut 370) respectively coupled to the pair of coupling parts.

The first and second coupling parts 310 and 320 have a hollow cylindrical shape, respectively have screw threads 330 and 340 on an inner perimeter surface, and respectively have opened grooves 380 and 390 for opening a side so that the connecting rod 200 or the vertebra fixing rod 400 is inserted thereto. The connecting unit 300 is combined to the connecting rod 200 and the vertebra fixing rod 400, thereby connecting the vertebra fixing rod 400 and the connecting rod 200.

Accordingly, the first coupling part 310 of the connecting unit 300 accommodates the vertebra fixing rod 400 and then fastens the first nut 360, and the second coupling part 320 accommodates the connecting rod 200 and then fastens the second nut 370, thereby fixing and mutually connecting the vertebra fixing rod 400 and the connecting rod 200.

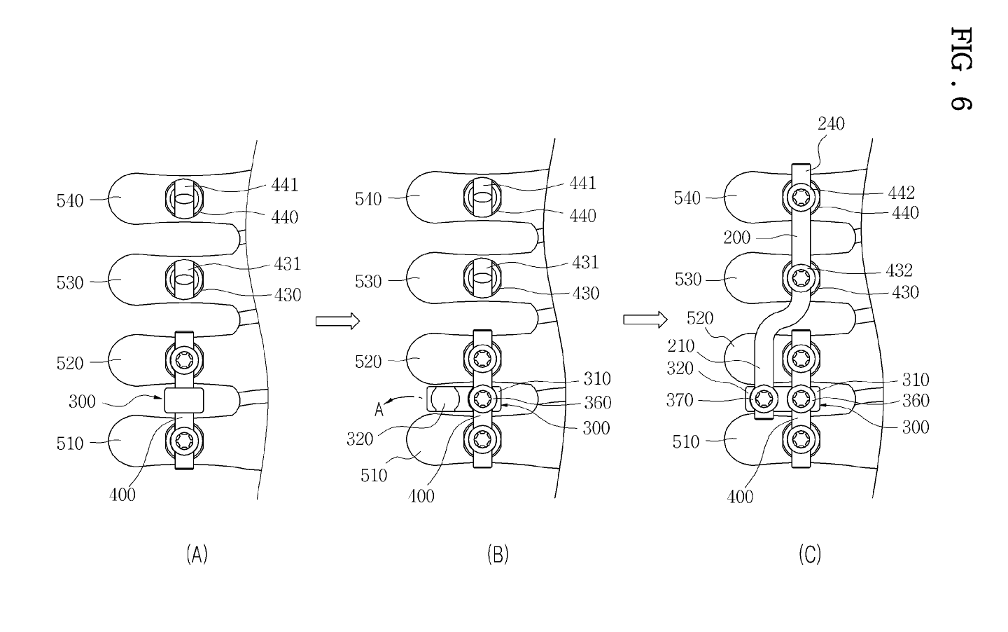

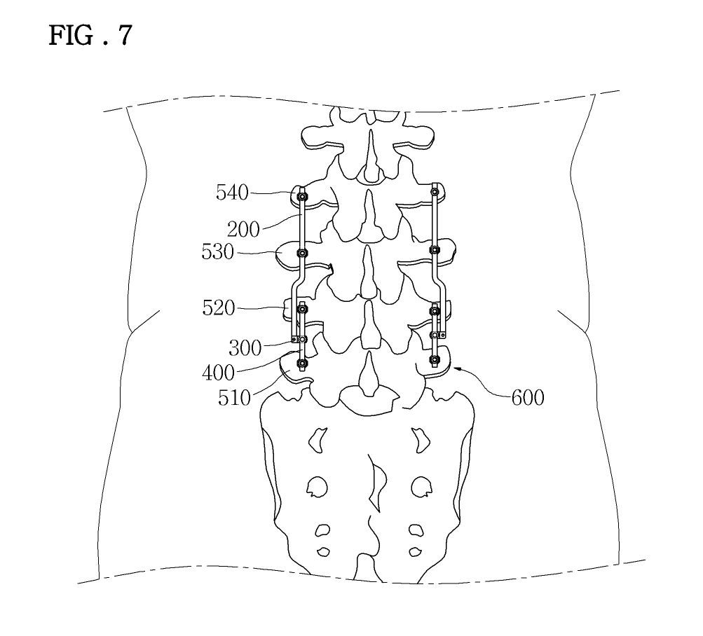

FIG. 6 is a diagram sequentially illustrating connecting processes of a spinal rod system, according to an embodiment of the present invention, and FIG. 7 is a diagram of a spinal rod system fixing vertebrae 600, according to an embodiment of the present invention.

In the vertebrae 600, when the vertebra fixing rod 400 is fixed to a first transverse process 510 and a second transverse process 520 via a prior procedure, a third transverse process 530 and a fourth transverse process 540 are damaged, a damaged vertebra may be fixed by being connected to the vertebra fixing rod 400 as follows.

As shown in FIG. 6(A), pedicle screws 430 and 440 are respectively implanted into the third and fourth transverse processes 530 and 540 of the damaged vertebra. Then, the connecting unit 300 is installed in the center of the vertebral fixing rod 400 fixing the first and second transverse processes 510 and 520 of the vertebrae 600 in such a way that the second coupling part 320 of the connecting unit 300 is implanted toward an inner part of a patient, and the first coupling part 310 is connected in a side direction of the vertebra fixing rod 400.

Here, the connecting unit 300 of FIG. 6(A) is installed as above so as to minimize an incision on the patient, and may not be installed as such. Accordingly, any method of installing the connecting unit 300 may be used as long as the first coupling part 310 of the connecting unit 300 is connected to the vertebra fixing rod 400.

Then, as shown in FIG. 6(B), the connecting unit 300 is rotated in a direction indicated by an arrow A so that the first and second coupling parts 310 and 320 both face a surgeon, and then the first nut 360 is coupled to the first coupling part 310 into which the vertebra fixing rod 400 is inserted, thereby binding the connecting unit 300 to the vertebra fixing rod 400.

Next, as shown in FIG. 6(C), the one end part 210 of the connecting rod 200 is inserted into the second coupling part 320 of the connecting unit 300, and the other end part 240 of the connecting rod 200 is mounted on head units 431 and 441 respectively of the pedicle screws 430 and 440 that are respectively implanted into the third and fourth transverse processes 530 and 540. Then, the one end part 210 of the connecting rod 200 is fixed to the connecting unit 300 by coupling the second unit 370 to the second coupling part 320, and the other end part 240 of the connecting rod 200 is fixed by coupling nuts 432 and 442 respectively to the head units 431 and 441 of the pedicle screws 430 and 440 implanted into the third and fourth transverse processes 530 and 540.

Accordingly, the damaged third and fourth transverse processes 530 and 540 of the vertebrae 600 are fixed by the connecting rod 200 connected to the pre-fixed vertebra fixing rod 400.

### INDUSTRIAL APPLICABILITY

According to a spinal rod system of the present invention having the above structure, an additionally damaged vertebra is simply and safely fixed by connecting the spinal rod system to a vertebra fixing rod fixing a vertebra through a prior procedure, and thus the vertebra is not strained, thereby reducing pain of a patient. Also, by reducing a number of pedicle screws additionally fixing the vertebra, a surgery time and efforts are reduced, thereby improving safety and reliability on a surgery.

1. A spinal rod system comprising:
a connecting rod comprising a first curved part and a second curved part so that one end part and another end part are spaced apart from each other and parallel to each other; and
a connecting unit for connecting the one end part of the connecting rod to a vertebra fixing rod fixed to a vertebra through a prior procedure.

2. The spinal rod system of claim 1, wherein the connecting unit comprises:
a pair of coupling parts formed in a single body to accommodate the connecting rod and the vertebra fixing rod; and
a pair of nuts coupled to the coupling part to fix the connecting rod and the vertebra fixing rod.
